(19) United States
(12) Patent Application Publication
(10) Pub. No.: US 2003/0215502 A1
(43) Pub. Date: Nov. 20, 2003

(54) Fast dissolving dosage forms having reduced friability

(75) Inventors: Karl Pruss, Bedminster, PA (US); Susan Wendel, Schwenksville, PA (US); Stephen Ruddy, Schwenksville, PA (US)

Correspondence Address: FOLEY AND LARDNER, SUITE 500, 3000 K STREET NW, WASHINGTON, DC, 20007, US

(73) Assignee: Elan Pharma International Limited

(21) Appl. No.: 10/392,383

(22) Filed: Mar. 20, 2003

Related U.S. Application Data
(60) Provisional application No. 60/365,526, filed on Mar. 20, 2002.

(51) Int. Cl. A61K 9/20; A61K 38/00; A61K 31/734; D04H 1/00; B27N 3/00

(52) U.S. Cl. 424/465; 514/2; 514/54; 514/179; 264/109

(57) ABSTRACT

Disclosed is a rapidly disintegrating or dissolving (fast melt) solid dosage form of at least one active agent and at least one pharmaceutically acceptable water-disintegrable or water-soluble excipient, wherein the dosage form has two opposed double-convex surfaces. The dosage form of the invention has the advantage of exhibiting low friability with a very low disintegration time.

### FIELD OF THE INVENTION

The present invention is directed to rapidly disintegrable dosage forms having very low friability, and to methods of making and using such dosage forms.

### BACKGROUND OF THE INVENTION

Rapidly disintegrating or dissolving dosage forms (collectively referred to herein as fast melt dosage forms) are useful for the rapid absorption, particularly buccal absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

To overcome such problems manufacturers have developed a number of fast melt solid dose oral formulations. These are available from manufacturers including Cima Labs, Fuisz Technologies Ltd., Prographarm, R. P. Scherer, Yamanouchi-Shaklee, and McNeil-PPC, Inc. All of these manufacturers market different types of rapidly dissolving solid oral dosage forms.

Cima Labs markets OraSolv®, which is an effervescent direct compression tablet having an oral dissolution time of five to thirty seconds, and DuraSolv®, which is a direct compression tablet having a taste-masked active agent and an oral dissolution time of 15 to 45 seconds. Cima's U.S. Pat. No. 5,607,697, for “Taste Masking Microparticles for Oral Dosage Forms,” describes a solid dosage form consisting of coated microparticles that disintegrate in the mouth. The microparticle core of Cima's patented oral dosage form has a pharmaceutical agent and one or more sweet-tasting compounds having a negative heat of solution wherein the sweet-tasting compound can be mannitol, sorbitol, a mixture of an artificial sweetener and menthol, a mixture of sugar and menthol, or methyl salicylate. The microparticle core is coated, at least partially, with a material that retards dissolution in the mouth and masks the taste of the pharmaceutical agent. The microparticles are then compressed to form a tablet. Cima's patent discloses that other excipients can also be added to the tablet formulation.

WO 98/46215 for “Rapidly Dissolving Robust Dosage Form,” assigned to Cima Labs, is directed to a hard, compressed, fast melt formulation having an active ingredient and a matrix of at least a non-direct compression filler and lubricant. A non-direct compression filler is typically not free-flowing, in contrast to a direct compression (DC grade) filler, and usually requires additionally processing to form free-flowing granules.

Cima also has U.S. patents and international patent applications directed to effervescent dosage forms (U.S. Pat. Nos. 5,503,846, 5,223,264, and 5,178,878) and tableting aids for rapidly dissolving dosage forms (U.S. Pat. Nos. 5,401,513 and 5,219,574), and rapidly dissolving dosage forms for water soluble drugs (WO 98/14179 for “Taste-Masked Microcapsule Composition and Methods of Manufacture”).

Fuisz Technologies, now part of BioVail, markets Flash Dose®, which is a direct compression tablet containing a processed excipient called Shearform®. Shearform® is a cotton candy-like substance of mixed polysaccharides converted to amorphous fibers. U.S. patents describing this technology include U.S. Pat. No. 5,871,781 for “Apparatus for Making Rapidly Dissolving Dosage Units;” U.S. Pat. No. 5,869,098 for “Fast-Dissolving Comestible Units Formed Under High-Speed/High-Pressure Conditions;” U.S. Pat. Nos. 5,866,163, 5,851,553, and 5,622,719, all for “Process and Apparatus for Making Rapidly Dissolving Dosage Units and Product Therefrom;” U.S. Pat. No. 5,567,439 for “Delivery of Controlled-Release Systems;” and U.S. Pat. No. 5,587,172 for “Process for Forming Quickly Dispersing Comestible Unit and Product Therefrom.”

Prographarm markets Flashtab®, which is a fast melt tablet having a disintegrating agent such as carboxymethyl cellulose, a swelling agent such as a modified starch, and a taste-masked active agent. The tablets have an oral disintegration time of under one minute (U.S. Pat. No. 5,464,632).

R. P. Scherer markets Zydis®, which is a freeze-dried tablet having an oral dissolution time of 2 to 5 seconds. Lyophilized tablets are costly to manufacture and difficult to package because of the tablets sensitivity to moisture and temperature. U.S. Pat. No. 4,642,903 (R. P. Scherer Corp.) refers to a fast melt dosage formulation prepared by dispersing a gas throughout a solution or suspension to be freeze-dried. U.S. Pat. No. 5,188,825 (R. P. Scherer Corp.) refers to freeze-dried dosage forms prepared by bonding or complexing a water-soluble active agent to or with an ion exchange resin to form a substantially water insoluble complex, which is then mixed with an appropriate carrier and freeze dried. U.S. Pat. No. 5,631,023 (R. P. Scherer Corp.) refers to freeze-dried drug dosage forms made by adding xanthan gum to a suspension of gelatin and active agent. Finally, U.S. Pat. No. 5,827,541 (R. P. Scherer Corp.) discloses a process for preparing solid pharmaceutical dosage forms of hydrophobic substances. The process involves freeze-drying a dispersion containing a hydrophobic active ingredient and a surfactant, in a non-aqueous phase; and a carrier material, in an aqueous phase.

Yamanouchi-Shaklee markets Wowtab®, which is a tablet having a combination of a low moldability and a high moldability saccharide. U.S. patents covering this technology include U.S. Pat. No. 5,576,014 for “Intrabuccally Dissolving Compressed Moldings and Production Process Thereof,” and U.S. Pat. No. 5,446,464 for “Intrabuccally Disintegrating Preparation and Production Thereof.”

Other companies owning rapidly dissolving technology include Janssen Pharmaceutica. U.S. patents assigned to Janssen describe rapidly dissolving tablets having two polypeptide (or gelatin) components and a bulking agent, wherein the two components have a net charge of the same sign, and the first component is more soluble in aqueous solution than the second component. See U.S. Pat. No. 5,807,576 for “Rapidly Dissolving Tablet;” U.S. Pat. No. 5,635,210 for “Method of Making a Rapidly Dissolving Tablet;” U.S. Pat. No. 5,595,761 for “Particulate Support Matrix for Making a Rapidly Dissolving Tablet;” U.S. Pat. No. 5,587,180 for “Process for Making a Particulate Support Matrix for Making a Rapidly Dissolving Tablet;” and U.S. Pat. No. 5,776,491 for “Rapidly Dissolving Dosage Form.”

Eurand America, Inc. has U.S. patents directed to a rapidly dissolving effervescent composition having a mixture of sodium bicarbonate, citric acid, and ethylcellulose (U.S. Pat. Nos. 5,639,475 and 5,709,886).

L.A.B. Pharmaceutical Research owns U.S. patents directed to effervescent-based rapidly dissolving formulations having a pharmaceutically active ingredient and an effervescent couple comprising an effervescent acid and an effervescent base (U.S. Pat. Nos. 5,807,578 and 5,807,577).

Schering Corporation has technology relating to buccal tablets having an active agent, an excipient (which can be a surfactant) or at least one of sucrose, lactose, or sorbitol, and either magnesium stearate or sodium dodecyl sulfate (U.S. Pat. Nos. 5,112,616 and 5,073,374).

Laboratoire L. LaFon owns technology directed to conventional dosage forms made by lyophilization of an oil-in-water emulsion in which at least one of the two phases contains a surfactant (U.S. Pat. No. 4,616,047). For this type of formulation, the active ingredient is maintained in a frozen suspension state and is tableted without micronization or compression, as such processes could damage the active agent.

Takeda Chemicals Inc., Ltd. owns technology directed to a method of making a fast dissolving tablet in which an active agent and a moistened, soluble carbohydrate are compression molded into a tablet, followed by drying of the tablets (U.S. Pat. No. 5,501,861).

Finally, Elan's U.S. Pat. No. 6,316,029, for “Rapidly Disintegrating Oral Dosage Form,” disclosed fast melt dosage forms comprising nanoparticulate active agents. Nanoparticulate compositions are also described in U.S. Pat. Nos. 5,145,684 for “Surface Modified Nanoparticles,” 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,187 for “Method of Grinding Pharmaceutical Substances;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles; ” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydropropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(-)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;“6,221,400 for “Methods of Treating Mammals Using Nanocrystalline Formulations of Human Immunodeficiency Virus (HIV) Protease Inhibitors;“6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions,” 6,375,986 for “Solid Dose Nanoparticulate Compositions Comprising a Synergistic Combination of a Polymeric Surface Stabilizer and Dioctyl Sodium Sulfosuccinate,” 6,428,814 for “Bioadhesive Nanoparticulate Compositions Having Cationic Surface Stabilizers;” 6,431,478 for “Small Scale Mill;” and 6,432,381 for “Methods for Targeting Drug Delivery to the Upper and/or Lower Gastrointestinal Tract,” all of which are specifically incorporated by reference. In addition, U.S. patent application No. 20020012675 A1, published on Jan. 31, 2002, for “Controlled Release Nanoparticulate Compositions,” describes nanoparticulate compositions, and is specifically incorporated by reference.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles,” all of which are specifically incorporated by reference.

Amorphous small particle compositions are described in, for example, U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.

Fast melt tablets as described in the prior art are generally characterized as having low disintegration times when exposed, for example, to the aqueous environment of a patient's mouth. These low disintegration times can be generally achieved through careful adjustment of a tablet formulation, such as by using highly porous excipients in the tablet formulations. Moreover, it is recognized in the art that when fast melt tablets are formed by compression techniques, it is necessary to use low compression forces so as to yield tablets that can disintegrate readily. Unfortunately, the resultant tablets thus prepared can suffer from high friability, and therefore cannot readily withstand typical manufacturing, handling, and packaging forces.

Conversely, tablets which have been subjected to high compression forces during manufacturing generally exhibit low friability, but require much longer times to disintegrate and are thus not suitable for circumstances where fast melt tablets are desirable. Therefore, prior art methods of making tablets having low disintegration times generally yield tablets of high friability. Consequently, it is necessary in these cases to employ special manufacturing, handling, and/or packaging techniques to prevent the breakage or fracturing of fast melt tablets prepared by these methods. These considerations significantly increase production costs. Alternatively, where fast melt tablets exhibiting both low friability and low disintegration times are desired, prior art methods generally rely upon careful selection and adjustment of tablet formulations to yield a tablet possessing such properties. Such methods and tablets are disclosed, for example, in WO 99/44580.

The art suggests that tablet shape can affect friability. See Lachman, L. L. et al., The Theory and Practice of Industrial Pharmacy (Lea & Febiger, Philadelphia, 1986); Tableting Specification Manual (American Pharmaceutical Association, Washington, D.C., 1990). For example, McNeil-PPC, Inc. owner of U.S. Pat. No. 6,270,790, teaches chewable tablets comprising at least one active ingredient, a water-disintegrable, compressible carbohydrate, and a binder which are compressed into a convex-shaped tablet. The tablets thus prepared exhibit a friability of less than 1% while those prepared with flat-faced beveled edges give rise to a higher friability.

It would be desirable to furnish fast melt tablets exhibiting very low friability and low disintegration times in a manner that avoids the prior art methods of having to carefully select and adjust the formulation of such tablets.

Thus, there is a need in the art for fast melt tablets having acceptably low friabilities and which also exhibit very low disintegration times so that the tablets may be processed with standard equipment and packaged in bulk. There is also a need to standardize the manufacture of low friability and low disintegration tablets that is independent of the formulation of the tablet. The present invention satisfies both of these needs.

### SUMMARY OF THE INVENTION

This invention is directed to the surprising and unexpected discovery that fast melt dosage forms (tablets) having double convex shapes give rise to very low friability and disintegration times. The tablets of this invention are thus amenable to conventional manufacturing and packaging techniques, and yet are fast dissolving or disintegrating such that rapid therapeutic delivery of an active agent may be readily achieved.

It is one object of the invention to provide a fast melt solid dose formulation with a friability of less than about 2%, comprising an active agent and at least one pharmaceutically acceptable water-soluble or water-dispersible excipient. The active agent can be coated or without a coating and may be in a crystalline, semi-crystalline, amorphous, or semi-amorphous form, or in a combination thereof. The active agent can be water soluble or poorly water soluble. Where the active agent is poorly water soluble, the active agent can have a nanoparticulate particle size.

The excipient functions to rapidly disintegrate or dissolve the solid dose matrix surrounding the active agent upon contact with saliva. The fast melt formulation is formed into a tablet having opposed double convex-shaped surfaces such that the major axis cup radius is about 100 to about 400% of the tablet diameter, while the minor axis cup radius is about 10 to about 50% of the tablet diameter.

Another object of the invention is to provide a method of making a fast melt solid dose oral formulation with low friability. The method comprises: (1) combining an active agent with at least one pharmaceutically acceptable water-soluble or water-dispersible excipient, and (2) forming a solid dose form of the resulting composition for oral administration. Additionally, one or more pharmaceutically acceptable excipients can be added to the composition for administration.

Yet another object of the present invention is to provide a method of treating a mammal, including a human, requiring the rapid onset of therapeutic activity by administering a fast melt dosage form of this invention.

It is to be understood that both the foregoing general description and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIGS. 1a and 1b are front and side views, respectively, of a double convex tablet having a major cup axis of 1.500 inches and a minor cup axis of 0.175 inches.

FIGS. 2a and 2b are front and side views, respectively, of a double convex tablet having a major cup axis of 1.680 inches and a minor cup axis of 0.112 inches.

FIGS. 3a and 3b are front and side views, respectively, of a double convex tablet having a major cup axis of 1.812 inches and a minor cup axis of 0.100 inches.

### DETAILED DESCRIPTION OF THE PREFERRED EMBODIMENTS

A. Fast Melt Dosage Form

The present invention relates to the unexpected and surprising discovery of new fast melt solid dosage forms that exhibit low friability. The solid dosage forms are provided in double convex-shaped tablets such that target hardnesses of about 2-17 kiloponds (KP), and friabilities of less than about 2%, are obtained. The fast melt solid dosage forms of the invention offer the benefit of rapid presentation of an active agent and rapid dissolution of the active agent in the oral cavity of a patient.

Fast melt compositions of the present invention, which combine rapid disintegration with rapid dissolution, reduce the delay in the onset of therapeutic action associated with prior known rapidly dissolving dosage forms of poorly soluble active agents. Further, the opportunity for buccal absorption of the poorly soluble active agent is enhanced with the present invention. Yet another advantage of the fast melt dosage forms of this invention is that the use of nanoparticulate active agent particles eliminates or minimizes the feeling of grittiness found with prior art fast melt formulations of poorly soluble active agents.

Rapid melt dosage forms dissolve or disintegrate rapidly in the patient's mouth without chewing or the need for water within a short time frame. Because of their ease of administration, such compositions are particularly useful for the specific needs of pediatrics, geriatrics, and patients with dysphagia. Rapidly dissolving dosage forms can be beneficial because of their ease of administration, convenience, and patient-friendly nature. It is estimated that 35% to 50% of the population, and in particular pediatric and geriatric patients, find it difficult to swallow tablets and hard gelatin capsules. Fast melt dosage forms eliminate the need to swallow a tablet or capsule. Moreover, fast melt dosage forms do not require the addition of water or chewing.

One advantage typically associated with fast melt dosage forms is a reduction of the time lag between administration of a dose and the physical presentation of the active ingredient. This lag time is usually associated with the break up of the dosage form and the distribution of the active ingredient thereafter. A second advantage of fast melt dosage forms is that the rapid presentation of the active agent in the mouth upon administration may facilitate buccal absorption of the active agent directly into the blood stream, thus reducing the first pass effect of the liver on the overall bloavailability of active ingredient from a unit dose. This second advantage is dramatically enhanced for the fast melt formulations of the invention, where the active agent is water soluble, or in the case of a poorly water soluble active agent where the nanoparticulate size of the poorly water soluble active agent enables rapid dissolution in the oral cavity.

The present invention is described herein using several definitions, as set forth below and throughout the application.

“About” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable nanoparticulate active agent particles, ‘stable’ means that active agent particles do not appreciably flocculate or agglomerate due to interparticle attractive forces or otherwise increase in particle size.

“Nanoparticulate active agents” refers to active agents having an effective average particle size of less than about 2 microns (i.e., 2000 nm).

“Conventional active agents or drugs” refers to non-nanoparticulate or solubilized active agents or drugs. Non-nanoparticulate active agents have an effective average particle size of greater than about 2 microns.

1. Disintegration Time, Friability, and Tablet Shape

Surprisingly, the fast melt dosage forms of the present invention exhibit a relatively high degree of tensile strength. Tensile strength is determined by the hardness, size, and geometry of the solid dose. This is significant because if a solid dose (i.e., a tablet) is too brittle it will crumble or fragment. Such brittle tablets can also be difficult and expensive to package. Thus, the ideal rapidly disintegrating solid oral dose should have a degree of tensile strength to allow ease of packaging while also being rapidly disintegrating upon administration.

The fast melt solid oral dosage form according to the present invention has a disintegration time of less than about 3 minutes upon addition to an aqueous medium. More preferably, the fast melt solid oral dosage form has a disintegration time upon addition to an aqueous medium of less than about 2 minutes, less than about 1 minute, less than about 45 seconds, less than about 30 seconds, less than about 20 seconds, less than about 15 seconds, less than about 10 seconds, or less than about 5 seconds.

The fast melt solid dosage form of the invention exhibits very low friability. Preferably, a fast melt tablet will have a friability of less than about 2%, preferably less than about 1.5%, and most preferably less than about 1.0.

It was unexpectedly and surprisingly discovered that, for a fast melt dosage form with a given mass and hardness, the friability of such a dosage form may be readily reduced by varying the surface concavity of the dosage form. Thus, the present invention provides for fast melt tablets exhibiting low friabilities obtained by a method which, in contrast to prior art methods, is not dependent upon careful selection of formulation ingredients to achieve the desired low friability.

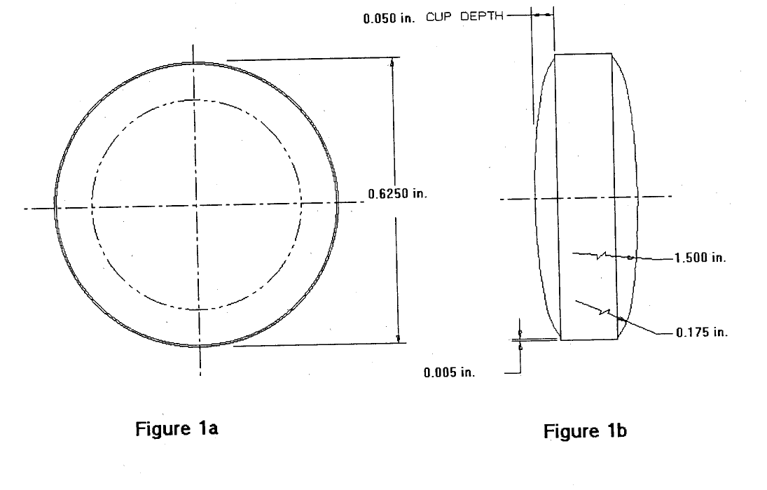

The fast melt dosage forms of the invention bear opposed, double convex cup faces. Referring to FIGS. 1b, 2b, and 3b, each face surface has two radii of curvature, R₁ and R₂. The radius of curvature R₁ at the portion of the face surface proximate to the edge of the tablet (minor axis cup radius) is about 5 to about 50% of the tablet diameter, and preferably about 16 to about 28% of the tablet diameter. The radius of curvature R₂ at the center of the tablet (major axis cup radius) is about 100 to about 400% of the tablet diameter, preferably about 240 to about 290% of the tablet diameter.

The shape of the tablet as viewed on its face is not limited to a circle, but encompasses any shape so long as the double convex face surfaces are maintained. A preferred embodiment of the invention is a tablet having a circular shape as viewed on its face. The diameters and masses of a tablet of the present invention may vary within ranges determined by a person who is skilled in the art, so long as the tablet maintains a friability of less than about 2% and a disintegration time of less than about 3 minutes.

2. Active Agent Generally

The starting composition (prior to formulation into a fast melt dosage form) comprises at least one active agent to be administered and at least one pharmaceutically acceptable excipient. Two or more active agents can be used in combination. An active agent can be a drug, therapeutic, pharmaceutical, or diagnostic agent, for example, a contrast agent, such as an x-ray contrast agent, or any other type of diagnostic material. Such agents include, for example, biologics such as proteins, peptides, and nucleotides. The active agent exists either as a discrete, crystalline phase, or as an amorphous phase. The crystalline phase differs from a non-crystalline or amorphous phase which results from precipitation techniques, such as those described in EP Patent No. 275,796. Two or more active agents can be used in combination.

The invention can be practiced with a wide variety of active agents. The active agent is preferably present in an essentially pure form. If the active agent has a nanoparticulate particle size, then the active agent is preferably poorly soluble and dispersible in at least one liquid dispersion medium. By “poorly soluble” it is meant that the active agent has a solubility in the liquid dispersion medium of less than about 30 mg/mL, less than about 20 mg/mL, less than about 10 mg/mL, or less than about 1 mg/mL. Useful liquid dispersion mediums include, but are not limited to, water, aqueous salt solutions, safflower oil, and solvents such as ethanol, t-butanol, hexane, and glycol.

The invention can be practiced with a wide variety of active agents. The active agent may be coated or without a coating. The active agent may be water soluble, or where it is poorly water soluble, the active agent can be in nanoparticulate form.

The active agent is preferably present in an essentially pure form and can be selected from a variety of known classes of agents, including, for example, proteins, peptides, nucleotides, anti-obesity drugs, nutraceuticals, dietary supplements, carotenoids, corticosteroids, elastase inhibitors, anti-fungals, oncology therapies, anti-emetics, analgesics, cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

Exemplary nutraceuticals and dietary supplements are disclosed, for example, in Roberts et al., Nutraceuticals: The Complete Encyclopedia of Supplements, Herbs, Vitamins, and Healing Foods (American Nutraceutical Association, 2001), which is specifically incorporated by reference. A nutraceutical or dietary supplement, also known as phytochemicals or functional foods, is generally any one of a class of dietary supplements, vitamins, minerals, herbs, or healing foods that have medical or pharmaceutical effects on the body. Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.”

A description of these classes of active agents and a listing of species within each class can be found in Martindale, The Extra Pharmacopoeia, Twenty-ninth Edition (The Pharmaceutical Press, London, 1989), specifically incorporated by reference. The active agents are commercially available and/or can be prepared by techniques known in the art.

Additionally, the fast melt compositions can be formulated to mask the unpleasant taste of an active agent. Such taste masking can be accomplished, for example, by the addition of one or more sweet tasting excipients or by coating the active agent particles with a sweet tasting excipient. Such taste masking is well-known in the art as described, for example, in U.S. Pat. No. 5,607,697.

3. Nanoparticulate Active Agent

In one embodiment of the present invention, the active agent has a nanoparticulate particle size. See e.g. U.S. Pat. No. 6,316,029. Nanoparticulate active agents preferably have an effective average particle size of less than about 2 microns, and at least one surface stabilizer associated with the surface of the active agent.

Nanoparticulate active agent compositions, first described in U.S. Pat. No. 5,145,684 (“the '684 patent”), are particles consisting of a poorly soluble active agent having adsorbed onto the surface thereof a surface stabilizer. The '684 patent describes the use of a variety of surface stabilizers for nanoparticulate compositions. The '684 patent also describes a method of screening active agents to identify useful surface stabilizers that enable the production of a nanoparticulate composition. Not all surface stabilizers will function to produce a stable, non-agglomerated nanoparticulate composition for all active agents.

Useful surface stabilizers, which are known in the art and described in the '684 patent, are believed to include those which physically associate with the surface of the active agent but do not chemically bond to or interact with the active agent. The surface stabilizer is associated with the surface of the active agent in an amount sufficient to maintain an effective average particle size of less than about 2000 nm for the active agent. Furthermore, the individual molecules of the surface stabilizer are preferably essentially free of intermolecular cross-linkages. Two or more surface stabilizers can be employed in the compositions and methods of the invention.

Suitable surface stabilizers can preferably be selected from known organic and inorganic pharmaceutical excipients. Such excipients include various polymers, low molecular weight oligomers, natural products, and surfactants. Surface stabilizers include nonionic, cationic, ionic, and zwitterionic surfactants.

Representative examples of surface stabilizers include gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminium silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation ), dialkylesters of sodium sulfosuccinic acid (e.g., Aerosol OT®, which is a dioctyl ester of sodium sulfosuccinic acid (American Cyanamid)); Duponol P®, which is a sodium lauryl sulfate (DuPont); Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-10G® or Surfactant 10-G® (Olin Chemicals, Stamford, Conn.); Crodestas SL-40® (Croda, Inc.); and SA90HCO, which is C18H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; random copolymers of vinyl pyrrolidone and vinyl acetate, such as Plasdone® S630, lysozyme, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™ (polyquaternium 10; Buckman Laboratories, Tenn.), tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ (quaternized ammonium salt polymers) and ALKAQUAT™ (benzalkonium chloride) (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

By “an effective average particle size of less than about 2000 nm” it is meant that at least 50% of the active agent particles have a particle size of less than about 2000 nm when measured by the above techniques. In other embodiments of the invention, at least about 70%, about 90%, about 95% or about 99% of the particles have a particle size less than the effective average particle size, i.e., less than about 2000 nm, less than about 1900 nm, less than about 1800 nm, etc. In yet other embodiments, the effective average particle size of the nanoparticulate composition is less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, or less than about 50 nm.

4. Pharmaceutically Acceptable Water-Soluble or Water-Dispersible Excipients

The pharmaceutically acceptable water-soluble or water-dispersible excipients are typically selected from a sugar, such as lactose, glucose, or mannose; a sugar alcohol, such as mannitol, sorbitol, xylitol, erythritol, lactitol, or maltitol; a starch or modified starch, such as corn starch, potato starch, or maize starch; a natural polymer or a synthetic derivative of a natural polymer, such as gelatin, carrageenin, an alginate, dextran, or maltodextran; a natural gum such as acacia or xanthan gum; a synthetic polymer, such as polyethylene glycol, polyvinylpyrrolidone, polyvinylalcohol, polyoxyethylene copolymers, polyoxypropylene copolymers, or polyethyleneoxide; or a mixture of any of these compounds. The pharmaceutically acceptable water-soluble or water-dispersible excipient can be a direct compression or a non-direct compression disintegrant.

5. Pharmaceutically Acceptable Excipients

Pharmaceutical compositions according to the invention may also comprise one or more binding agents, filling agents, lubricating agents, suspending agents, sweeteners, flavoring agents, preservatives, buffers, wetting agents, diluents, disintegrants, effervescent agents, glidants, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicifized microcrystalline cellulose (SMCC).

Suitable lubricating agents, including agents that act on the flowability of the powder to be compressed, include but are not limited to colloidal silicon dioxide, such as Aerosil® 200; talc, stearic acid, magnesium stearate, calcium stearate, sodium stearyl fumarate, such as PRUV®; and silica gel.

Examples of sweeteners are any natural or artificial sweetener, such as sucrose, xylitol, sodium saccharin, cyclamate, aspartame, and acsulfame.

Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol, such as Pearlitol SD200®; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone such as PVP XL®, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

6. Quantities of The Active Agent, Pharmaceutically Acceptable Excipients

The relative amount of the active agent in the fast melt formulations of the invention can vary widely and can depend upon, for example, the compound selected for delivery, the melting point of the compound, the water solubility of the compound, and the surface tension of water solutions of the compound. The active agent or pharmaceutically acceptable salt thereof may be present in any amount which is sufficient to elicit a desired effect and, where applicable, may be present either substantially in the form of one optically pure enantiomer or as a mixture, racemic or otherwise, of enantiomers.

The active agent can be present in the fast melt formulations of the invention in an amount of about 0.1% to about 99.9% (w/w), preferably about 5% to about 70% (w/w), and most preferably about 10% to about 50% (w/w), based on the total weight of the dry composition.

A pharmaceutically acceptable water-soluble or water-disintegrable excipient may be present in an amount of about 99.9% to about 0.1% (w/w), preferably about 95% to about 30% (w/w), and most preferably about 85% to about 60% (w/w), by weight based on the total weight of the dry composition. Typically, a diluent will be present in an amount of about 90% to about 10% (w/w); a disintegrant in an amount of about 20% to about 1% (w/w); a lubricant in an amount of about 3% to about 1% (w/w); and a glidant, if present, in an amount of about 5% to about 0.10% (w/w), by weight based on the total weight of the dry composition.

B. Methods of Making Fast Melt Solid Dose Compositions

Another embodiment of the invention relates to a method of preparing fast melt solid dose oral compositions that exhibit low friability. The method comprises: (1) providing a composition comprising the active agent and at least one pharmaceutically acceptable excipient; and (2) subjecting the composition to compression to form a solid dose form (e.g., tablet) of the composition having the required geometric features.

1. Active Agent Compositions

Compositions suitable for formation of the fast melt solid dose forms of the present invention may be first prepared by combining the desired amounts of active agent and at least one pharmaceutically acceptable water-disintegrable or water-soluble excipient. In some instances, it may desirable to first combine the active agent with one or more pharmaceutically acceptable excipients in a first mixture, and then adding the remaining pharmaceutically acceptable excipients in a second mixture, each separately blended prior to a final blending.

If desired, coated particles of the active agent can be used in this invention, for example, to mask an unpleasant taste of the active agent. Particles of the active agent should be coated with a layer of a coating agent having a thickness of about 3 to about 10 microns and substantially free of surface imperfections. Such coating agents include those previously mentioned as surface stabilizers for nanoparticulate active agents, and may be applied by conventional techniques known in the art using, for example, conventional fluidized bed coating equipment. The coated particles generally contain about 5 to about 60, preferably about 10 to 40, weight percent of the coating based on the total dry weight of the active agent, excipients, and coating.

In one embodiment, the active agent has a nanoparticulate particle size. Methods of making nanoparticulate compositions, which may comprise mechanical grinding, precipitation, homogenization, or any other suitable particle size reduction process, are known in the art and are described for example, in the '684 patent and in U.S. Pat. Nos. 5,518,187; 5,862,999; 5,718,388, and 5,510,118.

The one or more pharmaceutically acceptable water-soluble or water-dispersible excipients may be combined with a nanoparticulate active agent dispersion obtained after particle size reduction. The resultant composition can be blended and formulated into tablets for oral administration in the same manner as conventional particles. Alternatively, the nanoparticulate active agent dispersion can be spray dried or spray granulated, followed by blending with one or more pharmaceutically acceptable water-soluble or water-dispersible excipients, and tableting. The nanoparticulate active agent dispersion and desired excipients can be granulated to form a powder, followed by tableting. These methods are known in the art as described in U.S. Pat. No. 6,316,029, specifically incorporated herein by reference.

2. Blending of the Active Agent Compositions

The active agent and one or more pharmaceutically acceptable excipients can be blended to form a blend which may be directly compressed into tablets. The active agents and excipients need not be blended all together, but may be blended as separate mixtures that may then be combined and blended. The specific choice of ingredients for a particular blend, and the decision of whether to blend separate mixtures, are well within the purview of the skilled artisan.

An active agent can be blended with tablet excipients using any commercially available blending vessel known in the art. Exemplary blending vessels include a V-blender® (Blend Master Lab Blender, Patterson Kelley Co.) or high-shear mixer, Bohle bin, PK blenders, and blending bags. Depending upon the particular fast melt composition, blending times may vary between about 1 minute and 20 minutes.

A blend can also be prepared by lyophilizing a dispersion of a poorly soluble active agent and pharmaceutically acceptable excipients. Suitable lyophilization conditions include, for example, those described in EP 0,363,365 (McNeil-PPC Inc.), U.S. Pat. No. 4,178,695 (A. Erbeia), and U.S. Pat. No. 5,384,124 (Farmalyoc), all of which are incorporated herein by reference. Typically, the dispersion is placed in a suitable vessel and frozen to a temperature of between about −5° C to about -100° C. The frozen dispersion is then subjected to reduced pressure for a period of up to about 48 hours. The combination of parameters such as temperature, pressure, dispersion medium, and batch size will impact the time required for the lyophilization process. Under conditions of reduced temperature and pressure, the frozen solvent is removed by sublimation yielding a solid, porous, rapidly disintegrating solid oral dosage form having the active ingredient distributed throughout.

Alternatively, a blend can be prepared by granulating in a fluidized bed an admixture comprising the active agent and pharmaceutically acceptable excipients, to form a granulate. This is followed by tableting of the granulate to form a solid oral dosage form.

3. Tableting

The fast melt solid dose formulations of the present invention can be in the form of tablets for oral administration. The preparation of such tablets can be achieved through compression techniques known in the art using, for example, a single station tablet press, an automated tablet press, a rotary tablet press, or a high speed tablet press. The external force applied in the compression during the tableting step may be determined by the skilled artisan, so long as the resultant tablets exhibit friabilities of less than about 2% and disintegration times of less than about 3 minutes.

As mentioned previously, the shape of a tablet of the present invention as viewed on its face may be any shape known in the art. Exemplary shapes include triangle, square, round, animal-shape, irregular (caplet), ring (donut shape), and others such as those described in Tableting Specification Manual (American Pharmaceutical Association, Washington, D.C., 1990), which is specifically incorporated herein by reference.

In a preferred embodiment, a method is described by which circularly shaped tablets are prepared. In addition, letters or characters may be marked or applied to the tablets. Thus, the dies employed in the compression step of the tableting method may be readily adapted to any such shape, so long as the opposed cup surfaces of the tablet retain their double convex shapes.

C. Administration of Fast Melt Compositions

The present invention provides a method of treating a mammal, including a human, requiring the rapid availability of an active agent. The administered fast melt solid dosage form of the present invention rapidly releases an incorporated active agent resulting in fast onset of activity.

In general, the compositions of the invention will be administered orally to a mammalian subject in need thereof using a level of active agent that is sufficient to provide the desired physiological effect. The mammalian subject may be a domestic animal or pet but preferably is a human subject. The level of active agent needed to give the desired physiological result can be readily determined by one of ordinary skill in the art by referring to standard texts, such as Goodman and Gilman's and the Physician's Desk Reference.

The following examples are given to illustrate the present invention. It should be understood, however, that the invention is not to be limited to the specific conditions or details described in these examples. Throughout the specification, any and all references to a publicly available documents are specifically incorporated into this patent application by reference.

### EXAMPLES

### Example 1

Tablet Formulation

The purpose of this example was to prepare a fast melt dosage composition of an active agent.

Granules of the active agent were blended with mannitol and crospovidone in a blending vessel for about 10 minutes. Sodium stearyl fumarate and colloidal silicon dioxide were blended in a separate vessel for about 5 minutes, and then sieved through a 40 mesh screen. Both blends were combined and blended for about 3 minutes. The resulting mixture was used to prepare fast melt tablets having the composition shown in Table 1.

TABLE 1
Fast Melt Tablet Composition

| | | Composition | Batch | |
|---|---|---|---|---|
| | % | per Tablet | Formula (500 | |
| Ingredient | w/w | (mg) | Tablets) (g) | Functionality |
| Active Agent | 40.0 | 500.00 | 250.00 | Active |
| | | | | Pharmaceutical |
| | | | | Ingredient |
| mannitol (Pearlit- | 42.5 | 531.25 | 265.63 | Diluent |
| ol SD200 ®) | | | | |
| crospovidone | 15.0 | 187.50 | 93.75 | Super |
| (Plasdone XL ®) | | | | Disintegrant |
| sodium stearyl | 1.5 | 18.75 | 9.38 | Lubricant |
| fumarate | | | | |
| (PRUV ®) | | | | |
| colloidal silicon | 1.0 | 12.50 | 6.25 | Glidant |
| dioxide (Cab-O- | | | | |
| Sil M-5 ®) | | | | |
| Total | 100 | 1250 | 625.00 | |

### Example 2

Tablet Preparation

The purpose of this example was to form tablets of the composition prepared in Example 1 using a rotary tablet press (Riva Piccola®) under the tooling conditions given in Table 2. The tablets prepared in this example are shown in FIGS. 1a, 1b, 2a, 2b, 3a, and 3b as indicated in Table 2. For comparative purposes, a tablet having flat faces and beveled edges was also prepared under similar conditions. Tablets of the present invention exhibiting two different hardnesses thus resulted, along with standard tablets exhibiting the same hardnesses.

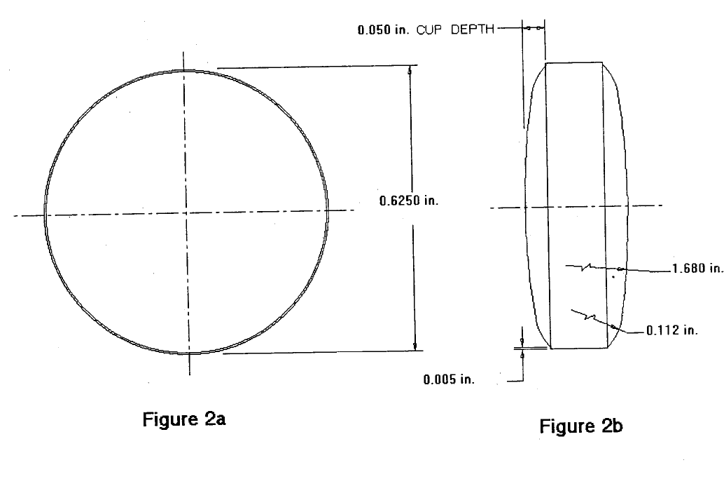

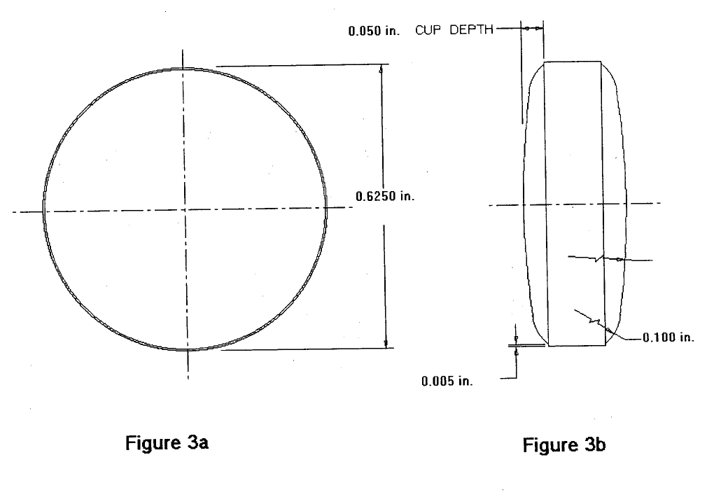

TABLE 2
Compression Force and Pressure of Fast Melt Composition

| | | | | | | | COMPARA- | |
|---|---|---|---|---|---|---|---|---|
| | | | | | | | TIVE TAB- | |
| | TABLET 1 | | TABLET 2 | | TABLET 3 | | LET 5/8″ | |
| | (FIGS. 1a | | (FIGS. 2a | | (FIGS. 3a | | Flat Face | |
| CONDITION | and 1b) | | and 2b) | | and 3b) | | Bevel Edge | |
| Hardness (KP) | 3 | 5 | 3 | 5 | 3 | 5 | 3 | 5 |
| Force (KN) | 11.3 | 13.5 | 10.6 | 13.5 | 10.5 | 13.1 | 9.4 | 11.3 |
| Applied | 57.07 | 68.18 | 53.53 | 68.18 | 53.03 | 66.16 | 47.47 | 57.07 |
| Pressure (Mpa) | | | | | | | | |

### Example 3

Hardness, Friability, and Disintegration Time

The purpose of this example was to evaluate the hardness, friability, and disintegration times of the tablets prepared in Example 2.

Tablets 1, 2, 3, and the comparative tablet were each prepared at hardnesses of 3 KP and 5 KP. Each of the tablets was then evaluated for friability and disintegration. Three tablets for each tablet shape were used for the data.

For the disintegration determination, a VanKel disintegration tester containing 710 micron sieves were used to test Tablets 1-3 and the comparative tablet in a 1000 mL deionized water bath at 37° C. Disintegration measurements were performed in accordance with USP 20. The friability results, together with disintegration times, are shown in Table 3.

TABLE 3
Friability and Disintegration Times for Fast Melt Composition

| | | | | | | | COMPARA- | |
|---|---|---|---|---|---|---|---|---|
| | | | | | | | TIVE TAB- | |
| | | | | | | | LET 5/8″ | |
| | TABLET 1 | | TABLE 2 | | TABLET 3 | | Flat Face | |
| PROPERTY | (FIG. 1) | | (FIG. 2) | | (FIG. 3) | | Bevel Edge | |
| Hardness (KP) | 3 | 5 | 3 | 5 | 3 | 5 | 3 | 5 |
| Friability at | 5.24 | 0.92 | 13.9 | 1.71 | 4.62 | 0.77 | 26.8 | 4.1 |
| 100 drops (% | | | | | | | | |
| Loss) | | | | | | | | |
| Disintegration | 10 | 14 | 12 | 15 | 13 | 15 | 15 | 17 |
| time (sec.) | 12 | 15 | 12 | 16 | 14 | 15 | 16 | 19 |
| | 13 | 15 | 12 | 17 | 15 | 17 | 17 | 20 |

Tablets 1-3 all exhibited friabilities of less than 14%. For tablets 1-3 prepared at a hardness of 3 KP, friability varied between 4.62 and 13.9% which is less than the friability of 26.8% for the comparative tablet. Similarly, the friability of Tablets 1-3 prepared at a hardness of 5 KP varied between 0.77 and 1.71%, which is less than the friability of 4.1% for the comparative tablet.

Tablets 1-3 all disintegrated in less than 15 seconds. These results demonstrate that, compared to a standard tablet, the disintegration times of the tablets of the present invention can be maintained at acceptable periods while the friabilities are substantially decreased, or at least maintained.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

We claim:

1. A rapidly disintegrating solid dosage form having opposed major face surfaces comprising:
(a) at least one active agent; and
(b) at least one pharmaceutically acceptable water-disintegrable or water soluble excipient,
wherein the dosage form: (i) substantially disintegrates or dissolves upon contact with an aqueous medium in less than about 3 minutes; (ii) has a friability of less than about 2%; and (iii) wherein each of the major face surfaces forms a double-convex shape.

2. The dosage form of claim 1, wherein the dosage form substantially disintegrates or dissolves upon contact with an aqueous medium in a time period selected from the group consisting of less than about 2 minutes, less than about 1minute, less than about 45 seconds, less than about 30 seconds, less than about 20 seconds, less than about 15 seconds, less than about 10 seconds, and less than about 5 seconds.

3. The dosage form of claim 1, wherein the friability is less than about 1%.

4. The dosage form of claim 1, wherein the double-convex surfaces have a major axis cup radius of about 100 to about 400% of the dosage form diameter, and have a minor cup radius of about 5 to about 50% of the dosage form diameter.

5. The dosage form of claim 4, wherein the major cup axis is about 240 to about 290% of the dosage form diameter and the minor cup axis is about 16 to about 28% of the dosage form diameter.

6. The dosage form of claim 1, wherein the concentration of the at least one active agent is selected from the group consisting of about 0.1% to about 99.9% (w/w), about 5% to about 70% (w/w), and about 20% to about 50% (w/w).

7. The dosage form of claim 1, wherein the concentration of the at least one pharmaceutically acceptable water-disintegrable or water-soluble excipient is selected from the group consisting of about 99.9% to about 0.1% (w/w), about 95% to about 30% (w/w), and about 85% to about 60% (w/w).

8. The dosage form of claim 1, wherein the at least one pharmaceutically acceptable water-disintegrable or water-soluble excipient is selected from the group consisting of a sugar, a sugar alcohol, a starch, a natural gum, a natural polymer, a synthetic derivative of a natural polymer, a synthetic polymer, and mixtures thereof.

9. The dosage form of claim 8, wherein the at least one pharmaceutically acceptable water-disintegrable or water-soluble excipient is selected from the group consisting of lactose, glucose, mannose, mannitol, sorbitol, xylitol, erythritol, lactitol, maltitol, corn starch, potato starch, maize starch, gelatin, carrageenin, an alginate, dextran, maltodextran, acacia gum, xanthan gum, polyethylene glycol, polyvinylpyrrolidone, polyvinylalcohol, polyoxyethylene copolymers, polyoxypropylene copolymers, polyethyleneoxide, and mixtures thereof.

10. The dosage form of claim 1, further comprising at least one pharmaceutically acceptable excipient selected from the group consisting of binding agents, lubricating agents, suspending agents, effervescent agents, diluents, and glidants.

11. The dosage form of claim 1, wherein the at least one active agent is in the form of crystalline particles, semi-crystalline particles, amorphous particles, semi-amorphous particles, or mixtures thereof.

12. The dosage form of claim 11, wherein the crystalline particles, semi-crystalline particles, amorphous particles, semi-amorphous particles, or mixtures thereof are coated with a coating agent.

13. The dosage form of claim 12, wherein the coating agent is present in an amount of about 5 to about 60% (w/w).

14. The dosage form of claim 1, wherein the at least one active agent:
(a) is poorly soluble;
(b) has an effective average particle size of less than about 2000 nm, and
(c) has at least one surface stabilizer associated with the surface of the active agent.

15. The dosage form of claim 14, wherein the composition has an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

16. The dosage form of claim 14, wherein the surface stabilizer is selected from the group consisting of an anionic surface stabilizer, a cationic surface stabilizer, an ionic surface stabilizer, and a zwitterionic surface stabilizer.

17. The dosage form of claim 14, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-,-β-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; random copolymers of vinyl acetate and vinyl pyrrolidone; and lysozyme.

18. The dosage form of claim 14, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl (C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

19. The dosage form of claim 1, wherein the at least one active agent is selected from the group consisting of proteins, peptides, nutraceuticals, carotenoids, anti-obesity agents, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products and substitutes, cardiac inotropic agents, contrast media, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, sex hormones, anti-allergic agents, stimulants and anoretics, sympathomimetics, thyroid agents, vasodilators, xanthines, acne medication, alpha-hydroxy formulations, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, and respiratory illness therapies associated with acquired immune deficiency syndrome.

20. A method of preparing a rapidly disintegrating solid dosage form, comprising the steps of:
(a) providing a composition comprising at least one active agent and at least one pharmaceutically acceptable water-disintegrable or water-soluble excipient; and
(b) forming a solid dosage form,
wherein the dosage form: (i) substantially disintegrates or dissolves upon contact with an aqueous medium in less than about 3 minutes; (ii) has a friability of less than about 2%; and (iii) wherein the dosage form has opposed major face surfaces that each form a double-convex shape.

21. The method of claim 20, wherein the dosage form substantially disintegrates or dissolves upon contact with an aqueous medium in a time period selected from the group consisting of less than about 2 minutes, less than about 1 minute, less than about 45 seconds, less than about 30 seconds, less than about 20 seconds, less than about 15 seconds, less than about 10 seconds, and less than about 5 seconds.

22. The method of claim 20, wherein step (a) comprises blending of the composition.

23. The method of claim 20, wherein step (b) comprises compression of the composition provided in step (a).

24. The method of claim 20, wherein the friability is less than about 1%.

25. The method of claim 20, wherein the surfaces have a major axis cup radius of about 100 to about 400% of the dosage form diameter, and have a minor cup radius of about 5 to about 50% of the dosage form diameter.

26. The method of claim 25, wherein the major cup axis is about 240 to about 290% of the dosage form diameter and the minor cup axis is about 16 to about 28% of the dosage form diameter.

27. The method of claim 20, wherein the concentration of the at least one active agent is selected from the group consisting of about 0.1% to about 99.9% (w/w), about 5% to about 70% (w/w), and about 20% to about 50% (w/w).

28. The method of claim 20, wherein the concentration of the at least one pharmaceutically acceptable water-disintegrable or water-soluble excipient is selected from the group consisting of about 99.9% to about 0.1% (w/w), about 95% to about 30% (w/w), and about 85% to about 60% (w/w).

29. The method of claim 20, wherein the at least one pharmaceutically acceptable water-disintegrable or water-soluble excipient is selected from the group consisting of a sugar, a sugar alcohol, a starch, a natural gum, a natural polymer, a synthetic derivative of a natural polymer, a synthetic polymer, and mixtures thereof.

30. The method of claim 29, wherein the at least one pharmaceutically acceptable water-disintegrable or water-soluble excipient is selected from the group consisting of lactose, glucose, mannose, mannitol, sorbitol, xylitol, erythritol, lactitol, maltitol, corn starch, potato starch, maize starch, gelatin, carrageenin, an alginate, dextran, maltodextran, acacia gum, xanthan gum, polyethylene glycol, polyvinylpyrrolidone, polyvinylalcohol, polyoxyethylene copolymers, polyoxypropylene copolymers, polyethyleneoxide, and mixtures thereof.

31. The method of claim 20, wherein the composition further comprises at least one pharmaceutically acceptable excipient selected from the group consisting of binding agents, lubricating agents, suspending agents, effervescent agents, diluents, and glidants.

32. The method of claim 20, wherein the at least one active agent is in the form of crystalline particles, semi-crystalline particles, amorphous particles, semi-amorphous particles, or mixtures thereof.

33. The method of claim 32, wherein the crystalline particles, semi-crystalline particles, amorphous particles, semi-amorphous particles, or mixtures thereof are coated with a coating agent.

34. The method of claim 33, wherein the coating agent is present in an amount of about 5% to about 60% (w/w).

35. The method of claim 20, wherein the at least one active agent:
(a) is poorly soluble;
(b) has an effective average particle size of less than about 2000 nm, and
(c) has at least one surface stabilizer associated with the surface of the active agent.

36. The method of claim 35, wherein the composition has an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

37. The method of claim 35, wherein the surface stabilizer is selected from the group consisting of an anionic surface stabilizer, a cationic surface stabilizer, an ionic surface stabilizer, and a zwitterionic surface stabilizer.

38. The method of claim 35, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; random copolymers of vinyl acetate and vinyl pyrrolidone; and lysozyme.

39. The method of claim 35, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

40. The method of claim 35, wherein the at least one active agent is selected from the group consisting of proteins, peptides, nutraceuticals, carotenoids, anti-obesity agents, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products and substitutes, cardiac inotropic agents, contrast media, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, sex hormones, anti-allergic agents, stimulants and anoretics, sympathomimetics, thyroid agents, vasodilators, xanthines, acne medication, alpha-hydroxy formulations, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, and respiratory illness therapies associated with acquired immune deficiency syndrome.

41. A method of treating a mammal comprising administering to the mammal an effective amount of a rapidly disintegrating solid dosage form, wherein the dosage form:
(a) comprises at least one active agent and at least one pharmaceutically acceptable water-disintegrable or water-soluble excipient;
(b) substantially disintegrates or dissolves upon contact with an aqueous medium in less than about 3 minutes;
(c) has a friability of less than about 1%; and
(d) has opposed major face surfaces that each form a double-convex shape.

42. The method of claim 41, wherein the dosage form substantially disintegrates or dissolves upon contact with an aqueous medium in a time period selected from the group consisting of less than about 2 minutes, less than about 1 minute, less than about 45 seconds, less than about 30 seconds, less than about 20 seconds, less than about 15 seconds, less than about 10 seconds, and less than about 5 seconds.

43. The method of claim 41, wherein the friability is less than about 1%.

44. The method of claim 41, wherein the surfaces have a major axis cup radius of about 100 to about 400% of the dosage form diameter, and have a minor cup radius of about 5 to about 50% of the dosage form diameter.

45. The method of claim 44, wherein the major cup axis is about 240 to about 290% of the dosage form diameter and the minor cup axis is about 16 to about 28% of the dosage form diameter.

46. The method of claim 41, wherein the concentration of the at least one active agent is selected from the group consisting of about 0.1% to about 99.9% (w/w), about 5% to about 70% (w/w), and about 20% to about 50% (w/w).

47. The method of claim 41, wherein the concentration of the at least one pharmaceutically acceptable water-disintegrable or water-soluble excipient is selected from the group consisting of about 99.9% to about 0.1% (w/w), about 95% to about 30% (w/w), and about 85% to about 60% (w/w).

48. The method of claim 41, wherein the at least one pharmaceutically acceptable water-disintegrable or water-soluble excipient is selected from the group consisting of a sugar, a sugar alcohol, a starch, a natural gum, a natural polymer, a synthetic derivative of a natural polymer, a synthetic polymer, and mixtures thereof.

49. The method of claim 48, wherein the at least one pharmaceutically acceptable water-disintegrable or water-soluble excipient is selected from the group consisting of lactose, glucose, mannose, mannitol, sorbitol, xylitol, erythritol, lactitol, maltitol, corn starch, potato starch, maize starch, gelatin, carrageenin, an alginate, dextran, maltodextran, acacia gum, xanthan gum, polyethylene glycol, polyvinylpyrrolidone, polyvinylalcohol, polyoxyethylene copolymers, polyoxypropylene copolymers, polyethyleneoxide, and mixtures thereof.

50. The method of claim 41, wherein the dosage form further comprises at least one pharmaceutically acceptable excipient selected from the group consisting of binding agents, lubricating agents, suspending agents, effervescent agents, diluents, and glidants.

51. The method of claim 41, wherein the at least one active agent is in the form of crystalline particles, semi-crystalline particles, amorphous particles, semi-amorphous particles, or mixtures thereof.

52. The method of claim 51, wherein the crystalline particles, semi-crystalline particles, amorphous particles, semi-amorphous particles, or mixtures thereof are coated with a coating agent.

53. The method of claim 52, wherein the coating agent is present in an amount of about 5 to about 60% (w/w).

54. The method of claim 41, wherein the at least one active agent:
(a) is poorly soluble;
(b) has an effective average particle size of less than about 2000 nm, and
(c) has at least one surface stabilizer adsorbed on the surface of the active agent.

55. The method of claim 54, wherein the nanoparticulate composition has an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

56. The method of claim 54, wherein the surface stabilizer is selected from the group consisting of an anionic surface stabilizer, a cationic surface stabilizer, an ionic surface stabilizer, and a zwitterionic surface stabilizer.

57. The method of claim 54, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; random copolymers of vinyl acetate and vinyl pyrrolidone; and lysozyme.

58. The method of claim 54, wherein the surface stabilizer is selected from the group consisting of cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

59. The method of claim 54, wherein the at least one active agent is selected from the group consisting of proteins, peptides, nutraceuticals, carotenoids, anti-obesity agents, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products and substitutes, cardiac inotropic agents, contrast media, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, sex hormones, anti-allergic agents, stimulants and anoretics, sympathomimetics, thyroid agents, vasodilators, xanthines, acne medication, alpha-hydroxy formulations, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, and respiratory illness therapies associated with acquired immune deficiency syndrome.
